(19) United States
(12) Patent Application Publication
(10) Pub. No.: US 2018/0318183 A1
(43) Pub. Date: Nov. 8, 2018

(54) SOFT-PACKAGED COSMETIC PREPARATION

(71) Applicant: BEIERSDORF AG, Hamburg (DE)

(72) Inventors: Esma ALANYA-ROUSSEAU, Hamburg (DE); Julia ECKERT, Hamburg (DE); Svea WISCHHOEFER, Hamburg (DE); Sabrina SCHULZE, Hamburg (DE); Christiane MEYER, Hamburg (DE); Anabelle VON DAVIER, Hamburg (DE); Frank HETZEL, Welle (DE)

(21) Appl. No.: 15/764,353

(22) PCT Filed: Sep. 19, 2016
(86) PCT No.: PCT/EP2016/072106

§ 371 Date: Mar. 29, 2018

(30) Foreign Application Priority Data
Oct. 2, 2015 (DE) 10 2015 219 066.8

(51) Int. Cl. A61K 8/06; A61K 8/81; A61Q 19/00

(52) U.S. Cl. CPC A61K 8/062; A45D 40/00; A61Q 19/00; A61K 8/81

(57) ABSTRACT

Cosmetic of a) a cosmetic preparation which contains one or more oils liquid at 20° C. and having a spreadability value of less than 550 mm/10 min and b) packaging of HDPE, LDPE or PP combined with a 1-propene-ethylene copolymer.

The present invention relates to a cosmetic comprising a cosmetic preparation containing one or more oils that are liquid at 20° C. and have a spreading value of less than 550 mm/10 min, and also a packaging made of HDPE, IDPE or PP in combination with 1-propene-ethylene copolymer.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (e.g. the appearance of fine lines and wrinkles).

Skincare products are generally stored in packaging in the form of bottles or jars. These predominantly consist of glass (glass jars) or plastics, in particular of purely high-density polyethylene (HDPE) or purely polypropylene. A disadvantage of such inherently very inexpensive and environmentally friendly packaging is the fact that the material is relatively stiff and inflexible, such that it regularly causes difficulties especially in removing high-viscosity preparations and in emptying the packaging as completely as possible.

Aside from bottles and jars, those skilled in the art are also familiar with the packaging form of metal or plastic tubes, the material of which is significantly softer and more flexible than is the case in bottles and jars. Plastic tubes are most commonly used in cosmetics due to their weight, their price, their corrosion resistance and the impression they make on consumers (such packaging with a “soft touch” feel is readily combined with preparations which convey a soft skin feel, so that the soft surface of the packaging emphasizes the impression of the product in terms of a holistic product concept). However, these plastic tubes have the disadvantage that, if the cosmetic preparation with which they are filled contains one or more oil components, they are not particularly storage-stable but rather, on contact with the oil, swell, since the oil is “sucked up” by the plastic and absorbed. Moreover, oil may separate out of the products. If nothing else, the oil in the plastic leads to lettering printed on the packaging being able to smudge and rub off. For these reasons, either only oil-free preparations (e.g. toothpaste) can be used to fill such packaging, or the packaging must be expensively coated or sealed on the inside which faces the cosmetic. Protective barriers made of ethylene vinyl alcohol, which are relatively expensive and less readily recyclable than purely polyolefin packaging, are known for example from the prior art.

It was therefore the object of the present invention to develop a cosmetic comprising an oil-containing cosmetic preparation and a soft-flexible (“soft-touch” feel) plastic packaging that is storage-stable, i.e. in which the oil absorption of the plastic packaging is significantly reduced, without it being necessary to apply a protective layer (e.g. made of ethylene vinyl alcohol) to the plastic.

The object is solved, surprisingly, by a cosmetic comprising

a) a cosmetic preparation containing one or more oils that are liquid at 20° C. and have a spreading value of less than 550 mm/10 min, and also

b) a packaging made of HDPE (high-density polyethylene), LDPE (low-density polyethylene) or PP (polypropylene) in combination with 1-propylene-ethylene copolymer.

All states of aggregation and temperature values are determined at standard pressure (1.013 bar).

The spreading value according to the invention is determined here as follows:

Measurement temperature: 20° C.,

Apparatus/auxiliaries: Eppendorf pipette (Multipette, VWR 613-3669), Eppendorf Combitips green (VWR no.: 613-2062), round Whatman 589/2 110 mm ashless/white ribbon filter paper (VWR 512-3608).

Procedure:

A filter paper is placed on the laboratory workbench (without any further support), the pipette is set to 25 μl and the lipid to be measured is taken up. The outside of the pipette is then wiped with a paper towel. 25 μl of the lipid to be measured are applied to the center of the filter paper from a height of approximately 30 mm and the exposure time is measured with a stopwatch. After 10 minutes of spreading time, the resulting spot is immediately drawn around with a pencil. This measurement is carried out on five filter papers for one lipid. The diameter in mm of the drawn-around spot is determined at three different points. In order to obtain the mean value, the 15 values (5 filter papers with 3 diameters each) are added together and then divided by 15. This mean value is then used to calculate the area of the spread lipid (calculation of the area of a circle):

$$A = π * {r}^{2} or A = π * {( \frac{d}{2} )}^{2}$$

The spreading value is given for 25 μl in mm²/10 min.

The following table gives some spreading values which were determined according to this method:

| | Spreading value |
|---|---|
| INCI | (mm²/10 min) |
| Olus Oil | 434 |
| Cocoglycerides | 460 |
| Octyldodecanol | 531 |
| Caprylic/Capric Triglyceride | 515 |
| Dicaprylyl Ether | 995 |
| C15-19 Alkane | 951 |
| Isopropyl Palmitate | 855 |
| Octocrylene | 113 |
| Decyl Oleate | 726 |
| Ethylhexyl Stearate | 647 |
| Isopropyl Stearate | 824 |
| Dimethicone | 353 |
| C12-15 Alkyl Benzoate | 750 |

It is advantageous according to the invention when the oils that are liquid at 20° C. and have a spreading value of less than 550 mm/10 min are selected from the group of compounds with the INCI names Olus Oil, Cocoglycerides, Octyldodecanol, Caprylic/Capric Triglyceride, Octocrylene, Dimethicone, Ethanol.

Moreover, embodiments of the present invention that are advantageous according to the invention are characterized in that the preparation is free from oils that are liquid at 20° C. selected from the group of compounds with the INCI names Dicaprylyl Ether, C15-19 Alkane, Isopropyl Palmitate, Decyl Oleate, Tridecyl Stearate, Tridecyl Trimellitate, Dipentaerythrityl Hexacaprylate/Hexacaprate, Ethylhexyl Stearate, Isopropyl Stearate, C12-15 Alkyl Benzoate. It is advantageous in the context of the present invention when the cosmetic preparation contains the oils that are liquid at 20° C. and have a spreading value of less than 550 mm/10 min in an amount of at least 1 wt %, based on the total weight of the preparation.

It is preferred in the context of the present invention when the cosmetic preparation contains the oils that are liquid at 20° C. and have a spreading value of less than 550 mm/10 min in an amount from 0.1 to 10 wt %, based on the total weight of the preparation.

It is advantageous according to the invention when Olus Oil is used in the preparation in an amount from 0.1 to 10 wt %, based on the total weight of said preparation.

It is advantageous according to the invention when cocoglycerides are used in the preparation in an amount from 0.1 to 10 wt %, based on the total weight of said preparation.

It is advantageous according to the invention when octyldodecanol is used in the preparation in an amount from 0.1 to 10 wt %, based on the total weight of said preparation.

It is advantageous according to the invention when caprylic/capric triglyceride is used in the preparation in an amount from 0.1 to 10 wt %, based on the total weight of said preparation.

It is advantageous according to the invention when Octocrylene is used in the preparation in an amount from 0.1 to 10 wt %, based on the total weight of said preparation.

It is advantageous according to the invention when dimethicone is used in the preparation in an amount from 0.1 to 10 wt %, based on the total weight of said preparation.

It is advantageous according to the invention when ethanol is used in the preparation in an amount from 0.1 to 10 wt %, based on the total weight of said preparation.

It is particularly preferred according to the invention when the preparation according to the invention contains a combination of ethanol and one or more oils from the group of the compounds Olus Oil, cocoglycerides, octyldodecanol, caprylic/capric triglyceride, octocrylene, dimethicone.

Embodiments of the present invention that are advantageous according to the invention are also characterized in that the cosmetic preparation contains glycerol.

In such a case, it is advantageous according to the invention when the content of glycerol is from 0.1 to 30 wt %, based on the total weight of the preparation.

It is further advantageous according to the invention when the cosmetic preparation is in the form of an emulsion. In this case, it is preferred according to the invention when the cosmetic preparation is in the form of an O/W emulsion.

Moreover, these emulsions are characterized as advantageous according to the invention in that the preparation contains one or more emulsifiers selected from the group of the compounds glyceryl stearate citrate, cetearyl alcohol, sodium cetearyl sulfate+glyceryl stearate, cetearyl sulfosuccinate, sodium stearoyl glutamate, polyglyceryl-3 methylglucose distearate, stearic acid, polyglyceryl-10 stearate, polyglyceryl-4 diisostearate/polyhydroxystearate/sebacate, potassium cetyl phosphate.

The concentration of emulsifier (total concentration) in this case is advantageously from 0.1 to 3 wt %, based on the total weight of the preparation.

Moreover, the inventive preparation may contain the ingredients that are customary for such compositions.

If the composition according to the invention contains one or more fillers such as, for example, silicates, calcite or magnesium carbonate, these fillers are present in an amount of at most 5 wt %, based on the total weight of the preparation.

It is further advantageous according to the invention when the preparation contains, as further ingredients, one or more compounds selected from the group of the compounds alpha-lipoic acid, folic acid, phytoene, D-biotin, coenzyme Q10, alpha-glucosylrutin, carnitine, carnosine, polidocanol, natural and/or synthetic isoflavonoids, flavonoids, creatine, creatinine, taurine, vitamin F, β-alanine, tocopheryl acetate, panthenol, almond oil, shea butter, cocoa butter, aloe vera, magnolol, honokiol, urea, hyaluronic acid, dihydroxyacetone, 8-hexadecene-1,16-dicarboxylic acid, glycyrrhetinic acid, glucosyl glycerides and/or licochalcone A.

Moreover, embodiments of the present invention that are advantageous according to the invention are characterized in that the preparation contains propylene glycol, butylene glycol, 2-methylpropan-1,3-diol, ethylhexylglycerol, 1,2-pentanediol, 1,2-hexanediol, 1,2-octanediol and/or 1,2-decanediol. In this case, a content of 2-methylpropan-1,3-diol, 1,2-pentanediol and/or 1,2-hexanediol is preferred according to the invention.

It is advantageous according to the invention when the preparation is free of parabens, isothiazolinones and 3-iodopmpargyl-N-butylcarbamate (IPBC).

It is also particularly advantageous according to the invention when the preparation according to the invention is free of sodium lauryl ether sulfate and cocamidopropyl betaines.

On the other hand, embodiments advantageous according to the invention are characterized in that the emulsion contains piroctone olamine (1-hydroxy-4-methyl-6-(2,4,4-trimethylpentyl)-2(1H)-pyridone monoethanolamine salt) and/or phenoxyethanol.

If the emulsion contains phenoxyethanol, it is advantageous according to the invention when the content of phenoxyethanol is from 0.1 to 1 wt %, based on the total weight of the emulsion.

It is advantageous according to the invention when the cosmetic preparation has a viscosity of greater than/equal to 2000 mPas/s. In this case, the viscosity according to the invention is determined as follows: Measurement at 25° C. in a 150 ml rolled rim bottle using Rheomat R123 from proRheo. The Rheomat R123 from proRheo GmbH is a rotational viscometer, i.e. a measurement body rotates in the substance to be measured. The force required to rotate the measurement body in the sample at a predefined speed is measured. From this torque, the speed of the measurement body and the geometric dimensions the measuring system used, the viscosity is calculated.

Measurement body no. 3 (item no. 200 0193), suitable for a viscosity range up to 1000 [mPAs], speed range 62.5 min.

Advantageously according to the invention, the pH of the preparation according to the invention is 4-9.

It is advantageous according to the invention when the packaging according to the invention is composed of two layers, wherein the inner layer which faces the preparation is formed of HDPE, LDPE or PP, while the outer layer is formed of a mixture of HDPE, LDPE or PP and in combination with 1-propene-1-ethylene copolymer. In this case, the same polymer (HDPE, LDPE or PP) of which the inner layer consists is always also used in the outer layer.

In such a case, it is preferred according to the invention when the combination of HDPE, LDPE or PP with 1-propene-ethylene copolymer in the outer layer has a mixture ratio (weight ratio) in which the content of 1-propene-ethylene copolymer is at least 50%. The mixture ratio of HDPE, LDPE or PP to 1-propene-ethylene copolymer is preferably for example of 40:60.

It is particularly preferred according to the invention when the combination of HDPE, LDPE or PP with 1-propene-ethylene copolymer in the outer layer is a mixture with a weight ratio of 50:50.

It is particularly preferred according to the invention in such a case when the two layers are formed in a weight ratio of 80 wt % inner layer and 20 wt % outer layer.

As HDPE (high-density polyethylene all commercially available polyethylenes of this group may be used.

As LDPE (low-density polyethylene), all commercially available polyethylenes of this group may be used.

As PP (polypropylene), all commercially available polypropylenes of this group may be used.

Advantageously according to the invention, the 1-propene-ethylene copolymer is a thermoplastic olefin.

It is advantageous according to the invention when this copolymer has a density (measured according to ISO 1183) of 0.89 g/cm³ (+/−10%).

It is moreover advantageous according to the invention when this copolymer has a melt flow rate at 230° C./2.16 kg (measured according to ISO 1133) of 0.80 g/cm³ (+/−10%).

It is further advantageous according to the invention when this copolymer has a tensile stress at yield (measured according to ISO 527-1, 527-2) of 9 MPa (+/−10%).

It is not least advantageous according to the invention when this copolymer has a tensile strain at break (measured according to ISO 527-1, 527-2) of 500% (+/−10%).

### Experiments

The following experiments made it possible to demonstrate the stability of the packaging toward the oils.

| | A | B |
|---|---|---|
| Composition in % | m [%] | m [%] |
| Glyceryl Stearate SE | 1.5 | 1.5 |
| Stearic Acid* | 2.5 | 2.5 |
| Cetyl Palmitate | 2.5 | 2.5 |
| Shea Butter | 1.0 | 1.0 |
| Myristyl Myristate | 2 | 2 |
| Octyldodecanol | | 10.00 |
| Dicaprylyl Ether | 10.00 | |
| Dimethicone | 1.0 | 1.0 |
| Carbomer | 0.2 | 0.2 |
| Glycerol | 5.0 | 5.0 |
| Sodium Hydroxide, q.s. to adjust pH | pH 6 | pH 6 |
| Phenoxyethanol | 0.90 | 0.90 |
| Cetearyl Alcohol | 3.0 | 3.0 |
| Water | to 100 | to 100 |
| Stability of formula after 1 month of | Swelling/deformation | Stable |
| storage at 40° C., packaging: | of the packaging | |
| LDPE in combination with 1-propene- | | |
| ethylene copolymer** | | |
| Stability of formula after 2 months of | Significant | Stable |
| storage at 40° C., packaging: | swelling/deformation | |
| LDPE in combination with 1-propene- | of the packaging | |
| ethylene copolymer** | | |

*Mixture of Palmitic Acid + Stearic Acid + Myristic Acid + Arachidic Acid + Oleic Acid
**= 2-layer structure, inner layer: LDPE, outer layer LDPE + 1-propene-ethylene copolymer in 50:50 weight ratio: Weight ratio of layers: 80% inner layer, 20% outer layer. 1-propene-ethylene copolymer: density: 0.89 g/cm³, melt flow rate: 0.80 g/cm³, tensile stress at yield: 9 MPa, tensile strain at break: 500%.

### EXAMPLES

The examples below are intended to illustrate the present invention without limiting it. All quantitative data, proportions and percentages are based on the weight and the total amount or on the total weight of the preparations.

| | C | D | E | F |
|---|---|---|---|---|
| Composition in % | m [%] | m [%] | m [%] | m [%] |
| Glyceryl Stearate SE | 1.5 | 1.5 | 1.5 | 1.5 |
| Stearic Acid* | 2.5 | 2.0 | 1.0 | 1.0 |
| Cetyl Palmitate | 2.5 | 2.5 | 2.0 | 2.0 |
| Shea Butter | 1.0 | 2.0 | 2.0 | 2.0 |
| Myristyl Myristate | 2 | 2 | | |
| Octyldodecanol | 10.00 | 6.00 | 2.00 | |
| Dimethicone | 1.0 | 0.5 | 2.0 | 2.0 |
| Caprylic/Capric Triglyceride | | | 4.0 | 8.0 |
| Carbomer | 0.2 | 0.2 | 0.2 | 0.2 |
| Glycerol | 5.0 | 8.0 | 8.0 | 8.0 |
| Phenoxyethanol | 0.90 | 0.70 | 0.70 | 0.70 |
| Cetearyl Alcohol | 3.0 | 3.0 | 2.0 | 2.0 |
| Ethylhexylglycerol | | 0.1 | 0.1 | 0.1 |
| Sodium EDTA | | 0.25 | 0.25 | 0.25 |
| Coenzyme Q10 | | | 1 | 1 |
| Ethanol | | | 3 | |
| Octocrylene | | | 1 | 1 |
| Butyl | | | 1 | 1 |
| Methoxydibenzoylmethane | | | | |
| Tapioca Starch | | | | 1 |
| Sodium Hydroxide, q.s. to | pH 5.5 | pH 6.0 | pH 7.0 | pH 7.1 |
| adjust pH | | | | |
| Water | to 100 | to 100 | to 100 | to 100 |
| Packaging | LDPE in | HDPE in | LDPE in | PP in |
| | combination | combination | combination | combination |
| | with 1- | with 1- | with 1- | with 1- |
| | propene- | propene- | propene- | propene- |
| | ethylene | ethylene | ethylene | ethylene |
| | copolymer** | copolymer** | copolymer** | copolymer** |

*Mixture of Palmitic Acid + Stearic Acid + Myristic Acid + Arachidic Acid + Oleic Acid
**= 2-layer structure, inner layer: polyolefin, outer layer polyolefin + 1-propene-ethylene copolymer in 50:50 weight ratio: Weight ratio of layers: 80% inner layer, 20% outer layer. 1-propene-ethylene copolymer: density: 0.89 g/cm³, melt flow rate: 0.80 g/cm³, tensile stress at yield: 9 MPa, tensile strain at break: 500%.

1.-15. (canceled)

16. A cosmetic, wherein the cosmetic comprises
(i) a cosmetic preparation comprising ethanol and/or one or more oils which are liquid at 20° C. and have a spreading value of less than 550 mm/10 min, and
(ii) a packaging made of HDPE, LDPE or PP in combination with ethylene-propylene copolymer.

17. The cosmetic of claim 16, wherein (i) comprises one or more substances selected from Olus Oil, cocoglycerides, octyldodecanol, caprylic/capric triglyceride, octocrylene, dimethicone, ethanol.

18. The cosmetic of claim 16, wherein (i) is free from dicaprylyl ether, C15-19 alkane, isopropyl palmitate, decyl oleate, tridecyl stearate, tridecyl trimellitate, dipentaerythrityl hexacaprylate/hexacaprate, ethylhexyl stearate, isopropyl stearate, and C12-15 alkyl benzoate.

19. The cosmetic of claim 17, wherein (i) is free from dicaprylyl ether, C15-19 alkane, isopropyl palmitate, decyl oleate, tridecyl stearate, tridecyl trimellitate, dipentaerythrityl hexacaprylate/hexacaprate, ethylhexyl stearate, isopropyl stearate, and C12-15 alkyl benzoate.

20. The cosmetic of claim 16, wherein (i) comprises the oils that are liquid at 20° C. and have a spreading value of less than 550 mm/10 min in a concentration of from 0.1 to 10 wt. %, based on a total weight of the preparation.

21. The cosmetic of claim 16, wherein (i) comprises the oils that are liquid at 20° C. and have a spreading value of less than 550 mm/10 min in a concentration of at least 1 wt. %, based on a total weight of the preparation.

22. The cosmetic of claim 16, wherein the cosmetic preparation further comprises glycerol.

23. The cosmetic of claim 16, wherein (i) is present as an emulsion.

24. The cosmetic of claim 23, wherein (i) is present as an O/W emulsion.

25. The cosmetic of claim 16, wherein (i) has a viscosity of at least 2000 mPas/s.

26. The cosmetic of claim 16, wherein (ii) is composed of two layers, wherein an inner layer which faces the preparation is formed of HDPE, LDPE or PP, and an outer layer is formed of a mixture of HDPE, LDPE or PP and ethylene-propylene copolymer, with the proviso that the same polymer (HDPE, LDPE or PP) of which the inner layer is formed is used in the outer layer.

27. The cosmetic of claim 26, wherein a weight ratio of HDPE, LDPE or PP and ethylene-propylene copolymer in the outer layer is 50:50.

28. The cosmetic of claim 26, wherein 80% inner layer and 20% outer layer are present.

29. The cosmetic of claim 27, wherein 80% inner layer and 20% outer layer are present.

30. The cosmetic of claim 16, wherein the ethylene-propylene copolymer has a density (measured according to ISO 1183) of 0.89 g/cm³ (+/−10%).

31. The cosmetic of claim 26, wherein the ethylene-propylene copolymer has a density (measured according to ISO 1183) of 0.89 g/cm³ (+/−10%).

32. The cosmetic of claim 16, wherein the ethylene-propylene copolymer has a melt flow rate at 230° C./2.16 kg (measured according to ISO 1133) of 0.80 g/cm³ (+/−10%).

33. The cosmetic of claim 26, wherein the ethylene-propylene copolymer has a melt flow rate at 230° C./2.16 kg (measured according to ISO 1133) of 0.80 g/cm³ (+/−10%).

34. The cosmetic of claim 16, wherein the ethylene-propylene copolymer has a tensile stress at yield (measured according to ISO 527-1, 527-2) of 9 MPa (+/−10%).

35. The cosmetic of claim 26, wherein the ethylene-propylene copolymer has a tensile stress at yield (measured according to ISO 527-1, 527-2) of 9 MPa (+/−10%).
